(19) United States
(12) Patent Application Publication
(10) Pub. No.: US 2021/0154388 A1
(43) Pub. Date: May 27, 2021

(54) TREATMENT SYSTEM, TREATMENT DEVICE AND CONSUMABLE FOR TREATMENT OF A PATIENT AS WELL AS A METHOD FOR PREPARING A TREATMENT SYSTEM

(71) Applicant: Fresenius Medical Care Deutschland GmbH, Bad Homburg (DE)

(72) Inventors: Lars Offermanns, Butzbach (DE); Christopher Hauke, Mainz-Kostheim (DE)

(21) Appl. No.: 17/047,981

(22) PCT Filed: Apr. 18, 2019
(86) PCT No.: PCT/EP2019/060086

§ 371 Date: Oct. 15, 2020

(30) Foreign Application Priority Data
Apr. 19, 2018 (DE) 10 2018 109 435.3

(51) Int. Cl. A61M 1/28; A61M 1/34

(52) U.S. Cl. CPC A61M 1/28; A61M 1/3413; A61M 2205/583; A61M 2202/0478; A61M 2205/6063; A61M 2205/582; A61M 2205/587

(57) ABSTRACT

The present disclosure pertains to a treatment system for treatment of a patient, in particular a treatment system for performing a hemodialysis, hemodiafiltration, and/or peritoneal dialysis. The system can include a treatment device, which comprises at least one seat for receiving a corresponding section of a consumable, and having a consumable received in the seat of the treatment device. The seat can comprise an alphanumerical identifier and the section of the consumable received in the seat can comprise an alphanumerical identifier matching the alphanumerical identifier of the corresponding seat.

### CROSS-REFERENCE TO RELATED APPLICATIONS

The present application is the national stage entry of International Patent Application No. PCT/EP2019/060086, filed on Apr. 18, 2019, and claims priority to Application No. DE 102018109435.3, filed on Apr. 19, 2018, the disclosures of which are expressly incorporated herein in their entirety by reference thereto.

### TECHNICAL FIELD

The disclosure relates to a treatment system for treatment of a patient, for example, a treatment system for performing a hemodialysis, hemofiltration, or peritoneal dialysis on a patient. The disclosure furthermore relates to a treatment device, for example a dialysis device, which may be used in the treatment system. The disclosure furthermore pertains to a consumable, for example, a tubing set or kit, which may be used in the treatment system having the treatment device. Further, the disclosure relates to a method for preparing the treatment system to prepare the treatment system for the treatment of a patient.

### BACKGROUND

Treatment systems for treatment of patients are known, for example, in the form of treatment systems for performing a dialysis treatment. In such treatment systems it is known that a treatment device in the form of a dialysis device is provided. The dialysis device typically comprises all the hardware requirements for performing a dialysis treatment on a patient. For example, the dialysis device may comprise pumps for pumping blood, a dialysis liquid, a substitute liquid and/or an anticoagulating agent. The dialysis device furthermore comprises clamps for the automatic opening and closing of fluid connections and sensors for monitoring pressures and temperatures. The dialysis device may furthermore comprise scales for determining liquid balances, mechanical holding devices for holding tubes, chambers or compartments, syringes, and dialysis filters. To be able to define and control the process or procedure of the dialysis treatment, control devices and input devices are furthermore generally provided, by which the dialysis treatment may be monitored and controlled for the entire course of treatment.

A dialysis device must generally be assembled with different consumables before the performance of the dialysis treatment. For example, a tubing set is inserted or received by various actuators to hence define the extracorporeal blood circuit and to feed the dialysis liquid through the dialysis filter.

To prepare the dialysis system for the treatment, for example, dripping chambers, syringes, and the dialysis filter, as examples of further consumables, are received by the dialysis device.

The consumables are typically formed as single use items or disposables, such that before the treatment of a patient, a respective new tubing set is inserted into the dialysis device to hence pros vide a hygienic and proper extracorporeal blood circuit in a simple manner. The tubes for providing the dialysis filter with the dialysis liquid are also provided as disposables. The dialysis filter itself is also to be understood as a disposable, wherein, depending on the configuration, it may be used for a single dialysis treatment or for multiple dialysis treatments, wherein generally for each dialysis patient, an individual dialysis filter is held available or reserved to ensure the hygiene and to avoid the transmittance of diseases.

In other words, for hygienic reasons, the consumables are either configured as a disposable or are at least individualized for each patient, such that the treatment device must be assembled with the respective individual consumables before the beginning of the treatment.

The assembly of the treatment devices, for example, in the form of dialysis devices, is a routine task, which is performed in dialysis centers as a standard by personnel. Since different dialysis devices, however, even in a single dialysis center, may be used next to or in proximity to each other, each dialysis device normally comprises an instruction manual to indicate in which manner the consumables must be attached or mounted to the treatment device for a particular treatment configuration. Such a manual may be present, for example, in the form of a step-by-step manual in paper or on a display of a dialysis device.

Accordingly, a person who assembles the dialysis device with the consumable before usage must concurrently keep the indicated manual in paper or on the display in sight, wherein the person looks from the manual to the actual device part or the seat in which the respective section of the consumable is to be received. For example, a particular tube section must be inserted into or received by a pump or a clamp. The person then looks back to the manual to verify whether did particular section of the consumable has actually been received by the correct seat to subsequently switch the display to a next step or to turn the pages of a paper manual to be provided with the next assembly step.

In a further embodiment, as disclosed, e.g., in DE 10 2011 004 620 B4, a signaling device is provided, by which the respective position on a treatment device is indicated at which an action or actuation by a user is required.

### SUMMARY

This disclosure describes treatment systems for a treatment of a patient, treatment devices, consumables and methods for preparing a treatment system for treatment of a patient, which enable an improved assembly.

In some embodiments, the treatment system are for performing a hemodialysis, hemofiltration, and/or peritoneal dialysis. Various embodiments are defined in the Figures, as well as the present description.

Accordingly, a treatment system for treatment of a patient, in particular a treatment system for performing a hemodialysis, hemodiafiltration, and/or peritoneal dialysis is described herein, wherein the treatment system comprises a treatment device, which comprises at least one seat for receiving a corresponding section of a consumable, and wherein a consumable is received in the seat of the treatment device. According to the present disclosure, the seat comprises an alphanumerical identifier and the section of the consumable received in the seat comprises an alphanumerical identifier matching the alphanumerical identifier of the corresponding seat.

Since both the seat and the section of the consumable to be received in the seat comprise a matching alphanumerical identifier or marking, the assembly of the treatment system is significantly simplified or facilitated.

A coworker may accordingly simply insert the consumable by inserting the section of the consumable having a respective alphanumerical identifier into the corresponding seat having the matching alphanumerical identifier, without reference or with reduced reference to a display or another instruction manual. Thereby, a correct matching between the section of the consumable to be received in a seat and the corresponding seat may be facilitated in a particular convenient manner.

In this way the operation safety may be improved since the respective user, which assembles the treatment system for the treatment of the patient, may focus on the treatment device and the consumable and is not additionally distracted by the indications on a display or in an external manual.

The assembly of the treatment device for the operational readiness of the treatment system by the insertion of the consumable in the respective seat of the treatment device may accordingly be achieved in a particular simple manner.

By means of the alphanumerical identifier of both the section of the consumable and the seat an unambiguous determination of the position of the treatment device at which the corresponding section of the consumable is to be inserted is obtained. Thereby, a particularly high operational safety is obtained. Since the user, which assembles the treatment system for the treatment of the patient, may immediately recognize whether the consumable is correctly mounted on or received by the treatment device.

For example, at least two seats having different alphanumerical identifiers may be provided and the consumable may comprise sections corresponding to the seats, wherein said sections comprise alphanumerical identifiers matching the alphanumerical identifiers of the respective seat.

By using an alphanumerical identifier it is also possible, for example, to define a sequence for the assembly of the sections of the consumable in the respective seats. Thereby, an assembly or mounting of the respective sections of the consumable in the corresponding seats may also be performed in the correct sequence to correctly perform the steps of the assembly of the treatment system for the treatment of a patient in this manner. Also in this case, it is easy for the user performing the respective assembly of the treatment system for the treatment of a patient to recognize the sequence since an identical identifier is present both in the seat and on the corresponding section of the consumable.

The alphanumerical identifiers of the seats may e.g., define an assembly sequence for the sections of the consumable having the matching alphanumerical identifiers.

For example, alphanumerical identifiers for the different sections of a first consumable may be used, for example, in the form of sections of a first tube, the alphanumerical identifiers A1, A2, A3, . . . may be used. For the different sections of a second consumable, which is to be inserted into the treatment device after the first consumable, for example, a second tube, a corresponding numerical identifier such as B1, B2, B3, . . . may be used to accordingly provide the sequence for the second consumable to insert the sections of the consumable corresponding to the respective seats.

In order to indicate the completion of the mounting of a consumable, it may be provided that the last section of the respective consumable, which is to be received by a corresponding seat, is provided with an identifier indicating the completion of the mounting. For example, the section of the consumable to be mounted at the end and the corresponding seat may be indicated by e.g., “A-end” or “Bend”. By the same token, the first section of the consumable which is to be received by a corresponding seat may be provided with an identifier indicating the mounting start. For example, the section of the consumable to be mounted first and the corresponding seat may e.g. be indicated with “A-start” or “B-start”.

The seats of the treatment device, which are provided with the alphanumerical identifiers, may e.g. be selected from the following list: blood pump, substitute pump, heparin pump, clamp, pressure sensor, holder, tubing holder.

However, various further seats, in particular, those that are provided in a medical device and in which a section of a consumable may be inserted, are possible.

The section of the consumable, which is provided with an alphanumerical identifier, may e.g. be selected from the following list: arterial tube or line, venous tube, dripping chamber, dialysis filter, syringe, heparin tube.

However, various further sections of consumables, which may be inserted in or received by seats of a medical device, are possible.

In some embodiments, all sections of the treatment device may be provided with different alphanumerical identifiers and the consumables may, depending on the configuration of the treatment system, comprise only a selection of the alphanumerical identifiers of all the seats to receive the consumable only in a selection of the seats.

Hereby, a particular configuration of the treatment system may be achieved by selecting the consumable with the corresponding identifier. In a hemodialysis system, e.g., for assembly according to a first configuration for a single needle operation a consumable having a different identifier may hence be used compared to an assembly according to a second configuration for a double needle operation. In other words, the user may effectively select the configuration of the treatment system by the selection of the consumable.

The alphanumerical identifier of the section of the consumable may be provided directly on the material, or in the material of the consumable, for example, by means of coloring or printing. The alphanumerical identifier may also be provided by applying a label or little flag to the corresponding section of the consumable.

Furthermore, it is possible to generate the alphanumerical identifier on the respective section of the consumable by application of a material for generating an embossed identifier. In this manner, also a haptic effect may be achieved in such a way that a user perceives a haptic feedback, for example, when following the surface of the consumable, for example, a tube, to hence be able to feel the section of the consumable that is to be inserted in a seat in a fast and reliable manner.

Furthermore, it is provided that the alphanumerical identifier on a consumable and/or the treatment device may be applied by photoluminescent color or paint, such that e.g., different photoluminescent colors of the alphanumerical identifier may be excited separately from each other by means of a frequency variable light source, and hence also a lighting up of the corresponding alphanumerical identifier is facilitated by means of the illumination of the consumable and preferably also the treatment device. Accordingly, it is also possible to cause an illumination of different alphanumerical identifiers by means of the provision of different light frequencies, such that, for example, different configurations of the treatment system may hence be provided by, e.g., providing that only particular alphanumerical identifiers on the consumable light up, which may then be received by the corresponding seats according to the selected configuration. Alternatively, the identifier on the consumable may be provided by the application of photoluminescent colors or pains in an arbitrary form, wherein the sequence of the mounting is defined by a particular color sequence, which has been indicated to the user.

The alphanumerical identifier on a consumable and/or on the treatment device may also be provided in the form of printable LEDs, wherein the current supply for the printable LEDs may then be provided by the treatment device itself. The energy supply may be provided, e.g., via induction.

On the treatment device the alphanumerical identifier of the seats may be provided via a known marketing or inscription by means of colors, for example, printing colors, to hence facilitate a cost efficient identifier.

The alphanumerical identifier of the seats of the treatment device, in which the respective sections of the consumable are inserted, may also be provided by a corresponding display in the seat or in proximity of the seat. Such a display may e.g., be provided in the form of an electronic ink (e-ink) to hence provide an improved readability, in particular for treatment situations, wherein a strong illumination is provided, for example, in an intensive care unit.

The alphanumerical identifier on the respective seat of the treatment device may also be provided by means of a projection, to hence also provide a process, such that the individual sections of the consumable may be inserted into the respective seats.

### BRIEF DESCRIPTION OF THE FIGURES

Embodiments of the inventions will be explained in more detail in the following descriptions of the Figures, in which:

FIG. 1 is a schematic view of a treatment device in the form of a hemodialysis device for performing a hemodialysis on a patient, and

FIG. 2 is a schematic view of a treatment system for a dialysis treatment of a patient in the form of a hemodialysis system, wherein a treatment device in the form of a hemodialysis device and consumables in the form of tubes, syringes, and dripping chambers and received in seats of the treatment device are shown.

### DETAILED DESCRIPTION

In the following, example embodiments are described with reference to the accompanying figures. In the Figures, like elements are denoted by identical reference numerals and repeated description thereof may be omitted in order to avoid redundancies.

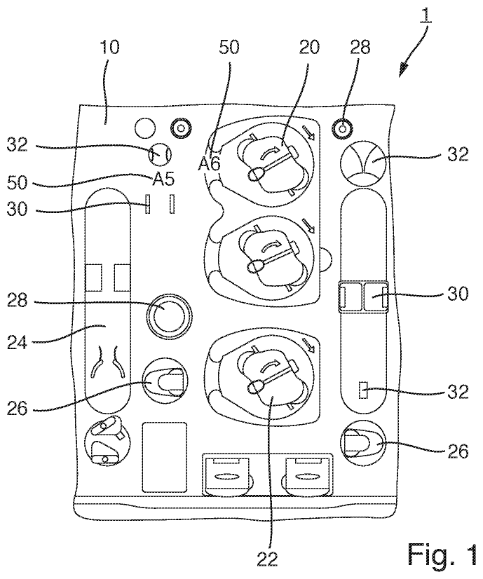

In FIG. 1, a schematic sectional view of a front side of a treatment device 10, which is here depicted as a hemodialysis device, is shown. The treatment device 10 is configured to enable the treatment of a patient and provides the instrument-based and control requirements for the performance of the treatment.

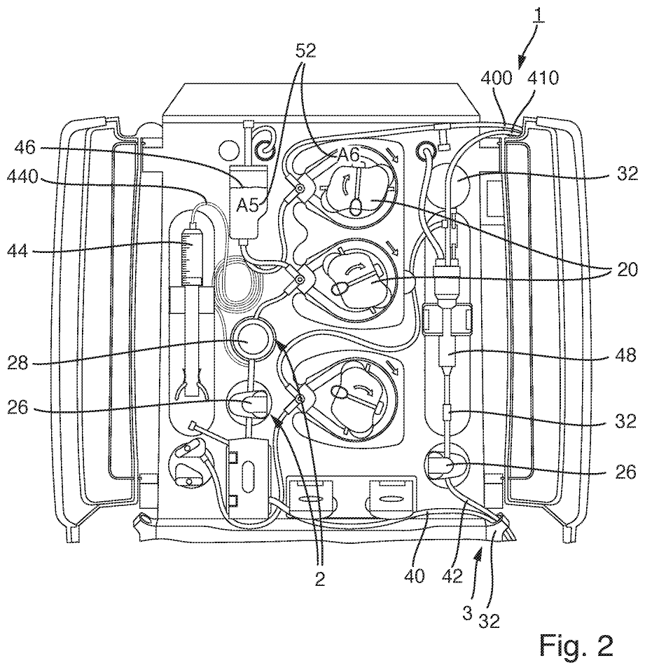

The treatment device 10 comprises a plurality of seats 2, 3, in which consumables 4 may be inserted, as shown in FIG. 2, to assemble the treatment device 10 for the performance of the treatment and to accordingly provide a treatment system 1. The treatment system 1 comprises, on the one hand, the treatment device 10 and, on the other hand, the consumables 4 received in the treatment device 10.

The sectional view of the treatment device 10, as depicted in FIGS. 1 and 2, is shown in the form of an extracorporeal blood module (EBM) of a hemodialysis device.

In the treatment device 10, in particular, actuators, clamps, holders, and sensors are provided, in which corresponding sections of the consumables 4 may be respectively inserted or received. The treatment device 10 of the treatment system 1 for treatment of a patient, as shown in FIG. 1, comprises, for example, a first and a second blood pump 20, a substitute pump 22, and a heparin pump 24 in the form of a syringe. Furthermore, actuators in the form of clamps 26 are provided, by which fluidic flows may be switched. In addition, pressure sensors 28 are provided, by which pressures may be monitored.

Aside from the pumps, clamps, and sensors, e.g., the seats 2 that are shown as exemplary active components of the treatment device 10, also passive components are provided in or on the treatment device 10, for example, passive seats 3 in the form of holders 30, which may serve to receive e.g., dripping chambers or pressure chambers or tube holders 32 for the attachment of sections of a tube.

In FIG. 2, the section of the treatment device 10 according to FIG. 1 is shown with inserted or received consumables 4. In this situation are status, the treatment system 1 is prepared for the treatment of a patient with regard to the hardware or instruments.

Herein, a consumable 4 is shown, e.g., in the form of an arterial tube or line 40, which e.g., is connected to a needle for the withdrawal of blood from the blood circuit of the patient to be treated, and in the form of a venous tube or line 42, which returns the blood treated in the treatment system 1, here the extracorporeal blood treatment module, again to the blood circulation of the treated patient.

Furthermore, a consumable 4 in the form of a heparin syringe 44 is provided, which is provided with a corresponding heparin tube 440. The heparin syringe 44 serves the purpose of feeding or providing an anticoagulant to the extracorporeal blood circuit to reduce or avoid a blood clotting in the extracorporeal blood circuit and in particular in the hemodialysis filter.

Further consumables 4 are provided in the form of dripping chambers 46, 48, which are provided for equalizing the pressure and to degas the blood.

A section of the arterial tube 40 and the venous tube 42 is respectively inserted in a seat 3 in the form of a tube holder 32.

Further sections of the consumable 4, for example, in the form of the arterial tube 40, are then received in further seats 2 in the form of active clamps 26, a pressure sensor 28, the first pump 20, and at a dripping chamber 46, in the perspective of the blood flow direction. Further sections of the consumable 4 are furthermore received or inserted, e.g., in the second pump 20 and are then provided in a tube section 400 to a dialysis filter, not shown.

A further tube section 410, which is returned from the dialysis filter, which is not shown, is received in a further seat 3 in the form of a tube holder 32 and is then provided to a dripping chamber 48, which is received in a seat 3 in the form of a holder 30. The consumables 4 extend from the dripping chamber 48 in the form of the venous tube 42. A section of the venous tube 42 is received in a further seat 3 in the form of the tube holder 32 and a further section of the venous tube 42 is received in a further seat 2 in the form of the clamp 26.

The configuration of the treatment system 1 is even more diverse than the above exemplary description. For example, consumables 4 in the form of feed lines to seats in the form of pressure sensors 28, as well as consumables 4 received in the seat in the form of the substitute pump 22 for the injection of the substitution liquid, are not further described.

The hemodialysis device, as shown in FIGS. 1 and 2, accordingly serves to perform a hemodialysis treatment of a patient. With the hemodialysis device, the treatment of a patient may be performed in large parts automatically, such that the interaction of the treatment personnel with the hemodialysis device is initially limited to the provision, assembly, and preparation of the hemodialysis system as well as the initiating and monitoring of the treatment.

To assemble the treatment system 1 for the treatment of a patient according to FIG. 2, the seats 2, 3 of the treatment device 10 must first be equipped with the correct consumables 4. Here, it is necessary that the sections of the consumables 4 are inserted in the respective correct positions and in the correct sequence in the seats 2, 3 of the treatment device 10.

As immediately evident from the exemplary configuration as shown, e.g., in FIG. 2, the tube guides are complex and it is absolutely to be taken care of that the accordingly provided sections of the tubes are inserted in the correct orientation and according to the correct sequence in the seats of the treatment device.

Therefore, it is required that the respective seats 2, 3 of the treatment device 10, which are provided in the shown exemplary embodiment, e.g., in the form of the mechanical holders 32 and 30, in the form of the pumps 20, 22, 24, in the form of sensors 28, as well as in the form of clamps 26, and the sensors 28, are each equipped with the correct section of the consumable 4, which are provided in the shown exemplary embodiment, e.g., in the form of sections of the tubes 40, 42, 400, 410, 440, in the form of the syringe 44, as well as in the form of dripping chambers 46, 48.

To achieve a correct association of the sections of the consumable 4 to the seats 2, 3, both the seats 2, 3 of the treatment device 10 and the respective corresponding sections of the consumable 4 are provided with corresponding alphanumerical identifiers.

By means of example, a consumable 4 is shown in the shown exemplary embodiment in the form of a dripping chamber 46, which is to be inserted into the seat 3 in the form of a mechanical holder 30. On the dripping chamber 46 an exemplary alphanumerical indication 52 has been applied. The alphanumerical identifier 52 is depicted as “A5”.

An alphanumerical identifier 50 is also provided on the seat 3, which is depicted as the mechanical holder 30, in which the dripping chamber 46 is to be inserted, wherein the alphanumerical identifier 50 is also depicted as “A5”. In other words, the alphanumerical identifier 50 of the seat 3 and the alphanumerical identifier 52 of the section of the consumable 4 match.

On the section of the consumable 4 in the form of the tube part, which is inserted or received in the upper pump 20, an alphanumerical identifier 52 is also applied, which is depicted as “A6”. This alphanumerical identifier 52 of the consumable 4 corresponds to an alphanumerical identifier 50, which is indicated on the upper pump 20 of the treatment device 10 as “A6”.

Accordingly, it is immediately recognizable for a user of the treatment system 1 for the treatment of a patient, when holding the consumable, e.g. the dripping chamber 46 and the tube set in his or her hands, which sections of the consumable are to be inserted in which seats of the treatment device.

The assembly sequence of the individual consumables or the individual sections of the consumables at the treatment device may hence be predefined by the alphanumerical identifier.

For example, the sections of the consumable, which are indicated with an “A”, are inserted in the corresponding seats before the sections of the consumable, which are e.g. indicated with a “B”, are inserted in the corresponding seats.

Furthermore, also the sequence within these structural parts may be further specified by e.g. performing a numerical progression of the assembly sequence. For example, starting from a section A1 of a first consumable, which accordingly is to be matched to a seat A1 of the treatment device, the section A2 of the consumable is to be matched with a seat A2 of the treatment device in a next step, and so on.

In this way, it is possible to intuitively perform an assembly of the treatment system in the correct sequence with the correct consumables only by reference to the consumable and the treatment device.

This is especially of significant importance, when a particular treatment system for the treatment of a patient is only rarely employed, as is the case, e.g., for particular devices, which are employed for the acute-care or intensive care medicine. Here, normally a particular treatment system for the treatment of the respective patient must be provided spontaneously under large pressure, and this treatment system is then prepared by the respective intensive care nurse or an assistant. By means of the simple matching of the respective alphanumerical identifiers on the consumable and the treatment device, an assembly of the respective treatment system may be accordingly provided in a fast, intuitive, and safe manner, without the necessity for the respective personnel to consult elaborate instruction manuals.

Although a dialysis device is described in the present disclosure, the inventive concepts described herein may also be transferred to various other medical devices, wherein consumables are to be received or inserted in treatment devices to prepare a treatment of a patient.

Where applicable, any possible combination and configuration of the described features as depicted in the exemplary embodiments can be chosen without leaving the scope of the disclosure.

### LIST OF REFERENCE NUMERALS

1 Treatment system for treatment of a patient
10 Treatment device
2 Seat
20 Blood pump
22 Substitute pump
24 Heparin pump
26 Clamp
28 Pressure sensor
3 Seat
30 Holder
32 Tube holder
4 Consumable
40 Arterial tube
42 Venous tube
44 Heparin syringe
400 Tube section
410 Tube section
440 Heparin tube
46 Dripping chamber
48 Dripping chamber
50 Alphanumerical identifier on the treatment device
52 Alphanumerical identifier on the consumable

1. A treatment system for performing a hemodialysis, hemodiafiltration, and/or peritoneal dialysis treatment of a patient, the treatment system having a treatment device which comprises a seat for receiving a corresponding section of a consumable, and having a consumable received in the seat of the treatment device de vice,
wherein the seat comprises an alphanumerical identifier, and wherein the section of the consumable received in the seat comprises an alphanumerical identifier matching the alphanumerical identifier of the corresponding seat.

2. The treatment system according to claim 1, wherein at least two seats having different alphanumerical identifiers are provided, and wherein the consumable comprises sections corresponding to the seats, said sections comprising alphanumerical numerical identifiers matching the alphanumerical identifiers of the respective seat.

3. The treatment system according to claim 2, wherein the alphanumerical identifiers of the seats define an assembly sequence for the sections of the consumable having the matching alphanumerical identifiers.

4. The treatment system according to claim 1, wherein the seat of the treatment device is selected from the group consisting of following list: a blood pump, a substitute pump, a heparin pump, a clamp, a pressure sensor, a holder, and a tubing holder.

5. The treatment system according to claim 1, wherein the section of the consumable is selected from the group consisting of: a tube, a venous tube, a dripping chamber, a dialysis filter, a syringe, and a heparin tube.

6. The treatment system according to claim 1, wherein all sections of the treatment device are provided with different alphanumerical identifiers and wherein the consumables, depending on the configuration of the treatment system, only comprise a selection of the alphanumerical identifiers of all the seats to receive the consumable only in a selection of the seats.

7. The treatment system according to claim 1, wherein characterized in that the alphanumerical identifier has been applied to the seat and/or the consumable with a photoluminescent color and/or wherein the alphanumerical identifier has been applied to the seat and/or the consumable with a material for generating an embossed identifier to provide a haptic feedback.

8. The treatment system according to claim 1, wherein in that the alphanumerical identifier is provided on a seat and/or on the consumable by a display, by a projection, or by printed LEDs.

9. The treatment system according to claim 2, wherein the seats are provided for receiving sections of the consumable.

10. The treatment system according to claim 9, wherein the alphanumerical identifiers of the seats define an assembly sequence for sections of the consumable having the matching alphanumerical identifiers.

11. The treatment system according to claim 9, wherein the seat of the treatment device is selected from the group consisting of: a blood pump, a substitute pump, a heparin pump, a clamp, a pressure sensor, a holder, and a tubing holder.

12. A consumable for use in the treatment system according to claim 1, wherein the consumable includes a section configured to be received by a seat of a treatment device, and wherein the section comprises an alphanumerical identifier.

13. The consumable according to claim 12, wherein the alphanumerical identifiers of the sections define an assembly sequence.

14. The consumable according to claim 12, wherein the section is selected from the group consisting of: an arterial tube, a venous tube, a dripping chamber, a dialysis filter, a syringe, and a heparin tube.

15. A method for preparing a treatment system for the treatment of a patient, wherein seats of a treatment device receive corresponding sections of a consumable, and wherein sections of the consumable comprise alphanumerical identifiers, which are matched to an alphanumerical identifier of the corresponding seats.
